(19) United States
(12) Patent Application Publication
(10) Pub. No.: US 2008/0194879 A1
(43) Pub. Date: Aug. 14, 2008

(54) Method for Producing an Ethylamine From Denatured Ethanol

(75) Inventors: Bram Willem Hoffer, Heidelberg (DE); Elmar Benne, Neu-Ulm (DE); Heinz Rutter, Kapellen (BE); Wolfgang Schlindwein, Karlsdorf-Neuthard (DE); Johann-Peter Melder, Bohl-Iggelheim (DE)

Correspondence Address: CONNOLLY BOVE LODGE & HUTZ, LLP, P O BOX 2207, WILMINGTON, DE, 19899, US

(73) Assignee: BASE SE, Ludwigshafen (DE)

(21) Appl. No.: 12/066,739

(22) PCT Filed: Sep. 6, 2006
(86) PCT No.: PCT/EP2006/066044

§ 371 Date: Mar. 13, 2008

(30) Foreign Application Priority Data
Sep. 13, 2005 (DE) 10 2005 043 440.1

(51) Int. Cl. C07C 209/14

(52) U.S. Cl. 564/479

(57) ABSTRACT

Processes comprising: (a) providing an ethanol reactant and ammonia; and (b) reacting the ethanol reactant with the ammonia in the presence of hydrogen and a heterogeneous hydrogenation/dehydrogenation catalyst to form a product comprising one or more ethylamines selected from the group consisting of monoethylamines, diethylamines, triethylamines and mixtures thereof; wherein the ethanol reactant comprises ethanol denatured by the addition of a denaturant comprising an ethylamine selected from the group consisting of diethylamine, triethylamine and mixtures thereof; and methods of denaturing ethanols.

The present invention relates to a process for preparing an ethylamine by reacting ethanol with ammonia, a primary amine or a secondary amine in the presence of hydrogen and a heterogeneous catalyst, the use of a novel agent for denaturing ethanol and the ethanol which has been denatured in this way.

Processes for preparing an ethylamine by reacting ethanol with ammonia, a primary amine or a secondary amine, in particular over transition metal catalysts, are known from the literature, cf., for example, Ullmann's Encyclopedia of Industrial Chemistry, 6th Edition, 2000 electronic release, “aliphatic Amines: Production from alcohols”.

The ethanol used can be produced synthetically, for instance by hydration of ethylene. An alternative to synthetic ethanol is ethanol prepared by biological or biochemical means, in particular by fermentation, known as bioethanol. This is prepared from renewable resources and is thus advantageous from an ecological point of view. In addition, bioethanol is sometimes cheaper than synthetic ethanol.

WO-A-05/063681 (BASF AG) relates to a process for preparing an ethylamine by reacting ethanol with ammonia, a primary amine or a secondary amine in the presence of hydrogen and a heterogeneous catalyst, in which process a biochemically prepared ethanol (bioethanol) in which the concentration of sulfur and/or sulfur-comprising compounds has been reduced beforehand by bringing it into contact with an adsorbent is used.

The German patent application number 102005012209.4 of Mar. 15, 2005 (BASF AG) describes a process for preparing an ethylamine by reacting ethanol with ammonia, a primary amine or a secondary amine in the presence of hydrogen and a heterogeneous hydrogenation/dehydrogenation catalyst, in which a biochemically or biologically prepared ethanol (bioethanol) is used, the catalyst comprises one or more metals of group VIII and/or IB of the Periodic Table and after activation by means of hydrogen has a CO uptake capacity of >100 μmol of CO/g of catalyst.

In certain cases, bioethanol has to be denatured by law. The term denaturation refers to making a substance which can be used as food/stimulant unpleasant, with it being used in another way in its denatured form. For this purpose, a denaturant which can be removed only with difficulty and/or has an unpleasant odor or taste is added to the substance. The object is to stop the substance which is utilized in another way from being used as food/stimulant, since when the substance is used as food/stimulant it is subject to higher tax than when used in another way. Alcohol is subjected to a distilled spirit tax and untaxed ethanol is therefore denatured.

Typical denaturants are MEK (methyl ethyl ketone) and Bitrex (denatonium benzoate, cf. www.bitrex.com) which has an extremely bitter taste. For vinegar production, the alcohol is denatured with acetic acid. For use as raw material in the chemical industry, shellac, toluene and cyclohexane, inter alia, are permitted as denaturants.

The distilled spirit regulations (EC) 1994 describe, for example, the following denaturants (cf. BrStV §30):
1. general:
   a) methyl ethyl ketone comprising from 95 to 96% by mass of MEK, from 2.5 to 3% by mass of methyl isopropyl ketone and from 1.5 to 2% by mass of ethyl isoamyl ketone (5-methyl-3-heptanone),
   b) shellac,
   c) spruce rosin,
   d) toluene,
   e) cyclohexane,
2. for producing cosmetic compositions or compositions for improving odor:
   a) diethyl phthalate,
   b) thymol,
   c) denatonium benzoate and tertiary butanol,
   d) isopropanol and tertiary butanol,
3. for producing scientific preparations for teaching purposes, for carrying out chemical studies of all types, for making up chemicals and reagents for in-house laboratory use, for the production, storage and sterilization of medical suture material and for producing sealing varnish:
   petroleum ether,
4. for producing emulsions and similar preparations for photographic purposes, phototype and blueprinting processes and for producing bandaging materials with the exception of collodium:
   ethyl ether,
5. for producing fuels:
   fuel,
6. for preparing ethyl tert-butyl ether (ETBE):
   ETBE,
7. for producing vinegar:
   acetic acid.

U.S. Pat. No. 2,176,208 (Christensen et al.) and U.S. Pat. No. 2,213,760 (Figg et al.) describe the denaturation of ethanol by means of a combination of chloroform and a primary amine.

It was an object of the present invention to discover an improved denaturant for ethanol, with the ethanol which has been denatured in this way advantageously being used, in particular, in a process for preparing an ethylamine, in particular monoethylamine, diethylamine and/or triethylamine.

On the basis of the disadvantages of the prior art recognized according to the invention, the denaturant should have the following properties:
1. It should trigger no undesirable chemical reactions when used in the synthesis of ethylamines; toluene could, for example, be hydrogenated and consume hydrogen.
2. It should not influence the catalyst when used in the ethylamine synthesis; sulfur components could, for example, poison the catalyst.
3. When used in the ethylamine synthesis, it must not interfere in the work-up of the products of the ethylamine synthesis, in particular not impair the quality of monoethylamine, diethylamine and triethylamine, and should be easy to separate off.
4. It must not accumulate when used in the ethylamine synthesis; unreacted ethanol is advantageously returned to the synthesis, so that traces of denaturant carried with it could accumulate in the process.
5. It has to be reusable. Since a relatively large amount of denaturant is generally necessary (typically, for example, up to 2% by weight), recyclability is absolutely necessary.

We have accordingly found a process for preparing an ethylamine by reacting ethanol with ammonia, a primary amine or a secondary amine in the presence of hydrogen and a heterogeneous hydrogenation/dehydrogenation catalyst, wherein an ethanol which has been denatured by addition of diethylamine and/or triethylamine is used.

Furthermore, we have found the use of diethylamine and/or triethylamine for denaturing ethanol and ethanol comprising diethylamine and/or triethylamine as denaturant.

In addition to the two individual ethylamines, it is also possible to use a binary mixture of the ethylamines as denaturant.

The process is particularly advantageous for preparing monoethylamine, diethylamine and/or triethylamine (MEA, DEA and/or TEA) by reacting the denatured ethanol with ammonia.

Advantages are:
1. the denaturant is readily available, in particular on site, (flexibility advantage),
2. no additional separation step is necessary (economic advantage),
3. the denaturant does not interfere in the synthesis (technical advantage).

Synthetic ethanol which can be used according to the invention preferably has a content of sulfur and/or sulfur-comprising compounds of ≦0.1 ppm by weight, e.g. from 0 to 0.07 ppm by weight, (in each case calculated as S), e.g. determined by the Wickbold method (DIN EN 41).

The bioethanol which can be used according to the invention is generally produced by fermentation from agrarian products such as molasses, sugarcane juice, maize starch or from products of saccharification of wood and from waste sulfite liquors.

Preference is given to using bioethanol which has been obtained by fermentation of glucose with elimination of CO₂ (K. Weissermel and H.-J. Arpe, Industrial Organic Chemistry, Wiley-VCH, Weinheim, 2003, p. 194; Electronic Version of Sixth Edition of Ullmann's Encyclopedia of Industrial Chemistry, 2000, Chapter Ethanol, Paragraph Fermentation).

The ethanol is generally isolated from the fermentation broths by distillation: Electronic Version of Sixth Edition of Ullmann's Encyclopedia of Industrial Chemistry, 2000, Chapter Ethanol, Paragraph ‘Recovery and Purification’.

Bioethanol used in the process of the invention can, for example, have a content of sulfur and/or sulfur-comprising compounds in the range from 0 to 50 ppm by weight, e.g. from 5 to 40 ppm by weight, (in each case calculated as S), e.g. determined coulometrically in accordance with DIN 51400 part 7.

In the process of the invention, it is also possible to use a biologically or biochemically prepared ethanol (bioethanol) in which the concentration of sulfur and/or sulfur-comprising compounds has been reduced beforehand, e.g. by bringing it into contact with an adsorbent, e.g. silica gel, an activated aluminum oxide, a zeolite having hydrophilic properties, an activated carbon or a carbon molecular sieve as described in WO-A-05/063681 and WO-A-05/063354 (both BASF AG).

For example, a bioethanol which has a content of sulfur and/or sulfur-comprising compounds in the range from 0 to 2 ppm by weight, particularly preferably from 0 to 1 ppm by weight, very particularly preferably from 0 to 0.5 ppm by weight, (in each case calculated as S), e.g. determined by the Wickbold method (DIN EN 41), can be used in the process of the invention.

The abovementioned sulfur-comprising compounds are inorganic compounds, e.g. sulfates, sulfites, and/or organic compounds, in particular symmetrical and/or unsymmetrical C₂₋₁₀-dialkyl sulfides, particularly preferably C₂₋₆-dialkyl sulfides, e.g. diethyl sulfide, di-n-propyl sulfide, diisopropyl sulfide, very particularly preferably dimethyl sulfide, C₂₋₁₀-dialkyl sulfoxides, e.g. dimethyl sulfoxide, diethyl sulfoxide, dipropyl sulfoxide, 3-methylthio-1-propanol, and/or S-comprising amino acids, e.g. methionine and S-methylmethionine.

If such a bioethanol is used in amination processes, the catalytically active metal surface of the respective hydrogenation catalyst becomes coated with sulfur or sulfur compounds to a lesser extent over time. This leads to a prolonged catalyst activity and thus to significantly better economics of the process.

The ethanol used, in particular in the process for preparing ethylamines, is preferably denatured by addition of from 0.01 to 50% by weight, particularly preferably from 0.1 to 20% by weight, very particularly preferably from 0.5 to 5% by weight, e.g. from 1 to 3% by weight, of diethylamine and/or triethylamine.

In one variant, the ethanol used, in particular in the process for preparing ethylamines, is denatured by the, preferably sole, addition of from 0.1 to 20% by weight, particularly preferably from 0.5 to 5% by weight, e.g. from 1 to 3% by weight, of diethylamine.

Particular preference is given to using no further additive in addition to diethylamine and/or triethylamine for denaturing ethanol.

The process of the invention for preparing ethylamines is preferably carried out continuously.

In a particularly preferred process variant, the reaction product after reaction of the ethanol with ammonia, which comprises monoethylamine, diethylamine and/or triethylamine, is fractionated by distillation and diethylamine and/or triethylamine obtained, in particular diethylamine, is used for denaturing ethanol used in the process.

The catalyst used in the process of the invention comprises one or more metals of group VIII and/or IB of the Periodic Table of the Elements.

Examples of such metals are Cu, Co, Ni and/or Fe, and also noble metals such as Ru, Pt, Pd, and also Re. The catalysts can be doped, for example, with Ag, Zn, In, Mn, alkali metals (Li, Na, Ka, Rb, Cs) and/or Mo.

As support material for these active metals, preference is given to using aluminum oxide (gamma, delta, theta, alpha, kappa, chi or mixtures thereof), silicon dioxide, zirconium dioxide, zeolites, aluminosilicates, etc, and also mixtures of these supports.

The catalysts can be produced by known methods, e.g. by precipitation, precipitation onto a support, impregnation.

The catalytically active composition of illustrative heterogeneous catalysts for the amination of the bioethanol used comprise, prior to treatment with hydrogen,

from 20 to 85% by weight, preferably from 20 to 65% by weight, particularly preferably from 22 to 40% by weight, of Al₂O₃, TiO₂, ZrO₂ and/or SiO₂,

from 1 to 30% by weight, particularly preferably from 2 to 25% by weight, of oxygen-comprising compounds of copper, calculated as CuO, and

from 14 to 70% by weight, preferably from 15 to 50% by weight, particularly preferably from 21 to 45% by weight, of oxygen-comprising compounds of nickel, calculated as NiO, with the molar ratio of nickel to copper preferably being greater than 1, in particular greater than 1.2, very particularly preferably from 1.8 to 8.5.

In a further variant, the catalytically active composition of these particularly preferred catalysts further comprises, prior to treatment with hydrogen,

from 15 to 50% by weight, particularly preferably from 21 to 45% by weight, of oxygen-comprising compounds of cobalt, calculated as CoO.

The oxygen-comprising compounds of copper, nickel and, if appropriate, cobalt, in each case calculated as CuO, NiO and CoO, in the preferred catalysts are generally comprised in the catalytically active composition (prior to treatment with hydrogen) in total amounts of from 15 to 80% by weight, preferably from 35 to 80% by weight, particularly preferably from 60 to 78% by weight, with the molar ratio of nickel to copper particularly preferably being greater than 1.

Further preferred heterogeneous catalysts for use in the process of the invention are

catalysts disclosed in DE-A-19 53 263 (BASF AG) which comprise cobalt, nickel and copper and aluminum oxide and/or silicon dioxide and have a metal content of from 5 to 80% by weight, in particular from 10 to 30% by weight, based on the total catalyst, with the catalysts comprising, calculated on the basis of the metal content, from 70 to 95% by weight of a mixture of cobalt and nickel and from 5 to 30% by weight of copper and with the weight ratio of cobalt to nickel being from 4:1 to 1:4, in particular from 2:1 to 1:2, for example the catalyst which is used in the examples there and has the composition 10% by weight of CoO, 10% by weight of NiO and 4% by weight of CuO on Al₂O₃,

catalysts which are disclosed in EP-A-382 049 (BASF AG) or can be prepared analogously whose catalytically active composition comprises, prior to treatment with hydrogen,

from 20 to 85% by weight, preferably from 70 to 80% by weight, of ZrO₂ andlor Al₂O₃,

from 1 to 30% by weight, preferably from 1 to 10% by weight, of CuO,

and in each case from 1 to 40% by weight, preferably from 5 to 20% by weight, of CoO and NiO, for example the catalysts described in loc. cit. on page 6 which have the composition 76% by weight of Zr, calculated as ZrO₂, 4% by weight of Cu, calculated as CuO, 10% by weight of Co, calculated as CoO, and 10% by weight of Ni, calculated as NiO,

catalysts disclosed in EP-A-963 975 (BASF AG) whose catalytically active composition prior to treatment with hydrogen comprises

from 22 to 40% by weight of ZrO₂,

from 1 to 30% by weight of oxygen-comprising compounds of copper, calculated as CuO,

from 15 to 50% by weight of oxygen-comprising compounds of nickel, calculated as NiO, with the molar Ni:Cu ratio being greater than 1,

from 15 to 50% by weight of oxygen-comprising compounds of cobalt, calculated as CoO,

from 0 to 10% by weight of oxygen-comprising compounds of aluminum and/or manganese, calculated as Al₂O₃ or MnO₂,

and no oxygen-comprising compounds of molybdenum,

for example the catalyst A disclosed in loc. cit., page 17, which has the composition 33% by weight of Zr, calculated as ZrO₂, 28% by weight of Ni, calculated as NiO, 11% by weight of Cu, calculated as CuO and 28% by weight of Co, calculated as CoO,

catalysts disclosed in EP-A-696 572 (BASF AG) whose catalytically active composition prior to reduction with hydrogen comprises from 20 to 85% by weight of ZrO₂, from 1 to 30% by weight of oxygen-comprising compounds of copper, calculated as CuO, from 30 to 70% by weight of oxygen-comprising compounds of nickel, calculated as NiO, from 0.1 to 5% by weight of oxygen-comprising compounds of molybdenum, calculated as MoO₃, and from 0 to 10% by weight of oxygen-comprising compounds of aluminum and/or manganese, calculated as Al₂O₃ or MnO₂, for example the catalyst disclosed in loc. cit, page 8, which has the composition 31.5% by weight of ZrO₂, 50% by weight of NiO, 17% by weight of CuO and 1.5% by weight of MoO₃,

catalysts described in EP A1-1 270 543 (BASF AG) which comprise at least one element or a compound of an element of groups VIII and IB of the Periodic Table

and

catalysts described in EP-A-1 431 273 (BASF AG) in whose production a precipitation of catalytically active components onto monoclinic, tetragonal or cubic zirconium dioxide has been carried out.

The catalysts produced can be stored as such. Before use as catalysts in the process of the invention, they are prereduced (=activation of the catalyst) by treatment with hydrogen. However, they can also be used without prereduction, in which case they are then reduced (=activated) by the hydrogen present in the reactor under the conditions of the process of the invention.

The catalyst is preferably activated by exposing it to a hydrogen-comprising atmosphere or a hydrogen atmosphere at a temperature in the range from 100 to 500° C., particularly preferably from 150 to 400° C., very particularly preferably from 180 to 300° C., for a period of at least 25 minutes, particularly preferably at least 60 minutes. The time for which the catalyst is activated can be up to 1 hour, particularly preferably up to 12 hours, in particular up to 24 hours.

During this activation, at least part of the oxygen-comprising metal compounds present in the catalysts are reduced to the corresponding metals, so that these are present together with the various oxygen compounds in the active form of the catalyst.

The process of the invention is suitable, for example, for preparing ethylamines of the formula I

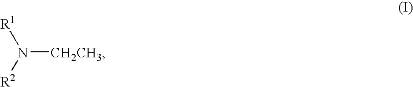

where
R¹, R² are each hydrogen (H), alkyl such as C₁₋₂₀₀-alkyl, cycloalkyl such as C₃₋₁₂-cycloalkyl, hydroxyalkyl such as C₁₋₂₀-hydroxyalkyl, aminoalkyl such as C₁₋₂₀-aminoalkyl, hydroxyalkylaminoalkyl such as C₂₋₂₀-hydroxyalkylaminoalkyl, alkoxyalkyl such as C₂₋₃₀-alkoxyalkyl, dialkylaminoalkyl such as C₃₋₃₀-dialkyl-aminoalkyl, alkylaminoalkyl such as C₂₋₃₀-alkylaminoalkyl, aryl, heteroaryl, aralkyl such as C₇₋₂₀-aralkyl or alkylaryl such as C₇₋₂₀-alkylaryl or together form —(CH₂)ⱼ—X—(CH₂)ₖ—,
X is CH₂, CHR³, oxygen (O), sulfur (S) or NR³,
R³ is hydrogen (H), alkyl such as C₁₋₄-alkyl, alkylphenyl such as C₇₋₄₀-alkylphenyl, and
j, k are each an integer from 1 to 4.

The process of the invention is therefore preferably employed for preparing an ethylamine I by reacting the bioethanol with a nitrogen compound of the formula II

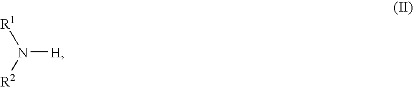

where R¹ and R² are as defined above.

Accordingly, in the preparation of the ethylamine I, a hydrogen atom of the nitrogen compound II is, purely formally, replaced by the radical CH₃CH₂— with liberation of one molar equivalent of water.

The substituents R¹ to R³, the variable X and the indices j, k in the compounds I and II have, independently of one another, the following meanings:

R¹, R²:
hydrogen (H),
alkyl such as C₁₋₂₀₀-alkyl, preferably C₁₋₂₀-alkyl, particularly preferably C₁₋₁₄-alkyl such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, n-hexyl, isohexyl, sec-hexyl, cyclopentylmethyl, n-heptyl, isoheptyl, cyclohexylmethyl, n-octyl, isooctyl, 2-ethylhexyl, n-decyl, 2-n-propyl-n-heptyl, n-tridecyl, 2-n-butyl-n-nonyl and 3-n-butyl-n-nonyl, in particular C₁₋₄-alkyl,
cycloalkyl such as C₃₋₁₂-cycloalkyl, preferably C₃₋₈-cycloalkyl such as cyclopropyl, cyclobutyl, cyclopentyl, cyclohexyl, cycloheptyl and cycloocyl, particularly preferably cyclopentyl and cyclohexyl,
hydroxyalkyl such as C₁₋₂₀-hydroxyalkyl, preferably C₁₋₈-hydroxyalkyl, particularly preferably C₁₋₄-hydroxyalkyl such as hydroxymethyl, 1-hydroxyethyl, 2-hydroxy-ethyl, 1-hydroxy-n-propyl, 2-hydroxy-n-propyl, 3-hydroxy-n-propyl and 1-(hydroxymethyl)ethyl,
aminoalkyl such as C₁₋₂₀-aminoalkyl, preferably C₁₋₈-aminoalkyl such as amino-methyl, 2-aminoethyl, 2-amino-1,1-dimethylethyl, 2-amino-n-propyl, 3-amino-n-propyl, 4-amino-n-butyl, 5-amino-n-pentyl, N-(2-aminoethyl)-2-aminoethyl and N-(2-aminoethyl)aminomethyl,
hydroxyalkylaminoalkyl such as C₂₋₂₀-hydroxyalkylaminoalkyl, preferably C₃₋₈-hydroxyalkylaminoalkyl such as (2-hydroxyethylamino)methyl, 2-(2-hydroxy-ethylamino)ethyl and 3-(2-hydroxyethylamino)propyl,
alkoxyalkyl such as C₂₋₃₀-alkoxyalkyl, preferably C₂₋₂₀-alkoxyalkyl, particularly preferably C₂₋₈-alkoxyalkyl such as methoxymethyl, ethoxymethyl, n-propoxy-methyl, isopropoxymethyl, n-butoxymethyl, isobutoxymethyl, sec-butoxymethyl, tert-butoxymethyl, 1-methoxyethyl and 2-methoxyethyl, particularly preferably C₂₋₄-alkoxyalkyl,
dialkylaminoalkyl such as C₃₋₃₀-dialkylaminoalkyl, preferably C₃₋₂₀-dialkylaminoalkyl, particularly preferably C₃₋₁₀-N,N-dialkylaminoalkyl such as (N,N-dimethylamino)methyl, (N,N-dibutylamino)methyl, 2-(N,N-dimethyl-amino)ethyl, 2-(N,N-diethylamino)ethyl, 2-(N,N-dibutylamino)ethyl, 2-(N,N-di-n-propylamino)ethyl, 2-(N,N-diisopropylamino)ethyl, (R³)₂N—(CH₂)_{q} (q=1 to 6), very particularly 3-(N,N-dimethylamino)propyl
alkylaminoalkyl such as C₂₋₃₀-alkylaminoalkyl, preferably C₂₋₂₀-alkylaminoalkyl, particularly preferably C₂₋₈-alkylaminoalkyl such as methylaminomethyl, 2-(methylamino)ethyl, ethylaminomethyl, 2-(ethylamino)ethyl and 2-(isopropyl-amino)ethyl, (R³)HN—(CH₂)_{q} (q=1 to 6),
aryl such as phenyl, 1-naphthyl, 2-naphthyl, 1-anthryl, 2-anthryl und 9-anthryl, preferably phenyl, 1-naphthyl und 2-naphthyl, particularly preferably phenyl,
heteroaryl such as 2-pyridinyl, 3-pyridinyl, 4-pyridinyl, pyrazinyl, pyrrol-3-yl, imidazol-2-yl, 2-furanyl and 3-furanyl,
aralkyl such as C₇₋₂₀-aralkyl, preferably C₇₋₁₂-phenylalkyl such as benzyl, p-methoxybenzyl, 3,4-dimethoxybenzyl, 1-phenethyl, 2-phenethyl, 1-phenyl-propyl, 2-phenylpropyl, 3-phenylpropyl, 1-phenylbutyl, 2-phenylbutyl, 3-phenyl-butyl and 4-phenylbutyl, particularly preferably benzyl, 1-phenethyl and 2-phenethyl,
alkylaryl such as C₇₋₂₀-alkylaryl, preferably C₇₋₁₂-alkylphenyl such as 2-methyl-phenyl, 3-methylphenyl, 4-methylphenyl, 2,4-dimethylphenyl, 2,5-dimethylphenyl, 2,6-dimethyiphenyl, 3,4-dimethylphenyl, 3,5-dimethylphenyl, 2,3,4-trimethyl-phenyl, 2,3,5-trimethylphenyl, 2,3,6-trimethylphenyl, 2,4,6-trimethylphenyl, 2-ethylphenyl, 3-ethylphenyl, 4-ethylphenyl, 2-n-propylphenyl, 3-n-propylphenyl and 4-n-propylphenyl,
or two radicals together form a —(CH₂)ⱼ—X—(CH₂)ₖ— group such as —(CH₂)₃—, —(CH₂)₄—, —(CH₂)₅—, —(CH₂)₆—, —(CH₂)₇—, —(CH₂)—O—(CH₂)₂—, —(CH₂)—NR³—(CH₂)₂—, —(CH₂)—CHR³—(CH₂)₂—, —(CH₂)₂—O—(CH₂)₂—, —(CH₂)₂—NR³—(CH₂)₂—, —(CH₂)₂—CHR³—(CH₂)₂—, —CH₂—O—(CH₂)₃—, —CH₂—NR³—(CH₂)₃—,

R³:
hydrogen (H),
alkyl, particularly C₁₋₄-alkyl such as methyl, ethyl, n-propyl, isopropyl, n-butyl, iso-butyl, sec-butyl and tert-butyl, preferably methyl and ethyl, particularly preferably methyl,
alkylphenyl, particularly C₇₋₄₀-alkylphenyl such as 2-methylphenyl, 3-methyl-phenyl, 4-methylphenyl, 2,4-dimethylphenyl, 2,5-dimethylphenyl, 2,6-dimethyl-phenyl, 3,4-dimethylphenyl, 3,5-dimethylphenyl, 2-, 3-, 4-nonylphenyl, 2-, 3-, 4-decylphenyl, 2,3-, 2,4-, 2,5-, 3,4-, 3,5-dinonylphenyl, 2,3-, 2,4-, 2,5-, 3,4- and 3,5-didecylphenyl,

X:
CH₂, CHR³, oxygen (O), sulfur (S) or NR³, preferably CH₂, NH and O,

j:
an integer from 1 to 4 (1, 2, 3 or 4), preferably 1 and 2, and

k:
an integer from 1 to 4 (1, 2, 3 or 4), preferably 1 and 2.

As aminating agent in the hydrogenative amination of bioethanol in the presence of hydrogen, it is possible to use either ammonia or primary or secondary, aliphatic or cycloaliphatic or aromatic amines.

When ammonia is used as aminating agent, the alcoholic hydroxyl group is firstly converted into the primary amino group (—NH₂). The primary ethylamine formed in this way can react with further bioethanol to form the corresponding secondary amine (diethylamine) and this can in turn react with further alcohol to form the corresponding tertiary amine (triethylamine). Depending on the composition of the reaction batch, or the feed stream (in the case of continuous operation) and depending on the reaction conditions employed, viz. pressure, temperature, catalyst, reaction time (space velocity over the catalyst), primary, secondary or tertiary ethylamines can be prepared preferentially as desired in this way.

Like ammonia, primary or secondary amines can be used as aminating agents.

These aminating agents are preferably used for preparing symmetrically substituted dialkylamines or trialkylamines, e.g. ethyldiisopropylamine and ethyldicyclohexylamine.

For example, the following monoalkylamines and dialkylamines are used as aminating agents: methylamine, dimethylamine, ethylamine, diethylamine, n-propylamine, di-n-propylamine, isopropylamine, diisopropylamine, isopropylethylamine, n-butylamine, di-n-butylamine, s-butylamine, di-s-butylamine, isobutylamine, n-pentylamine, s-pentyl-amine, isopentylamine, n-hexylamine, s-hexylamine, isohexylamine, cyclohexylamine, aniline, toluidine, piperidine, morpholine and pyrrolidine.

Amines which are particularly preferably prepared by the process of the invention are, for example, monoethylamine (from ethanol and ammonia), diethylamine (from ethanol and monoethylamine), triethylamine (from ethanol and diethylamine), monoethylamine/diethylamine/triethylamine mixture (from ethanol and ammonia) and dimethylethylamine (from ethanol and dimethylamine).

The aminating agent can be used in stoichiometric, substoichiometric or superstoichiometric amounts based on the alcoholic hydroxyl group to be aminated.

In the case of amination with primary or secondary amines, the amine is preferably used in an approximately stoichiometric amount or slightly superstoichiometric amount per mole of alcoholic hydroxyl group.

Ammonia, specifically, is generally used in a from 1.5- to 250-fold, preferably from 2- to 100-fold, in particular from 2- to 10-fold, molar excess per mole of alcoholic hydroxyl group to be reacted.

Higher excesses both of ammonia and of primary or secondary amines are possible.

The process of the invention can be carried out batchwise or preferably continuously as follows, with the catalyst preferably being located in the reactor as a fixed bed. However, the embodiment as a fluidized-bed reaction with upward and swirling motion of catalyst material is likewise possible.

The amination can be carried out in the liquid phase or in the gas phase. Preference is given to the fixed-bed process in the gas phase.

When working in the liquid phase, the starting materials (alcohol plus ammonia or amine) are simultaneously passed in the liquid phase at pressures of generally from 5 to 30 MPa (50-300 bar), preferably from 5 to 25 MPa, particularly preferably from 15 to 25 MPa, and temperatures of generally from 80 to 300° C., preferably from 120 to 270° C., particularly preferably from 130 to 250° C., in particular from 170 to 230° C., including hydrogen over the catalyst which is usually located in a fixed-bed reactor which is preferably heated from the outside. Both downflow mode operation and upflow mode operation are possible. The space velocity of the catalyst is generally from 0.05 to 5, preferably from 0.1 to 2, particularly preferably from 0.2 to 0.6, kg of alcohol per liter of catalyst (bed volume) and hour. If appropriate, the starting materials can be diluted with a suitable solvent such as tetrahydrofuran, dioxane, N-methylpyrrolidone or ethylene glycol dimethyl ether. It is advantageous to heat the reactants, preferably to the reaction temperature, before they are introduced into the reaction vessel.

When working in the gas phase, the gaseous starting materials (alcohol plus ammonia or amine) are passed in a gas stream which is sufficiently large for vaporization, preferably hydrogen, at pressures of generally from 0.1 to 40 MPa (1 to 400 bar), preferably from 0.1 to 10 MPa, particularly preferably from 0.1 to 7 MPa, in the presence of hydrogen over the catalyst. The temperatures for the amination are generally from 80 to 300° C., preferably from 120 to 270° C., particularly preferably from 160 to 250° C. Flow into the fixed catalyst bed can be either from above or from below. The gas stream required is preferably obtained by means of a gas recycle mode of operation.

The space velocity of the catalyst is generally in the range from 0.01 to 2, preferably from 0.05 to 0.5, kg of alcohol per liter of catalyst (bed volume) and hour.

The hydrogen is generally fed into the reaction in an amount of from 5 to 400 l, preferably in an amount of from 50 to 200 l, per mole of alcohol component, with the amount in liters in each case being based on standard conditions (S.T.P.).

It is possible to employ higher temperatures and higher total pressures both when working in the liquid phase and when working in the gas phase. The pressure in the reaction vessel, which is made up of the sum of the partial pressures of the aminating agent, the alcohol and the reaction products formed and also, if appropriate, the solvent which is concomitantly used at the indicated temperatures, is advantageously increased to the desired reaction pressure by injection of hydrogen.

The excess aminating agent can be circulated together with the hydrogen both in continuous operation in the liquid phase and in continuous operation in the gas phase.

If the catalyst is present as a fixed bed, it can be advantageous in terms of the selectivity of the reaction to mix, i.e. “dilute”, the shaped catalyst bodies in the reactor with inert packing elements. The proportion of packing elements in such catalyst preparations can be from 20 to 80 parts by volume, particularly preferably from 30 to 60 parts by volume and in particular from 40 to 50 parts by volume.

The water of reaction formed in the course of the reaction (in each case one mole per mole of alcohol group reacted) generally does not have an adverse effect on the degree of conversion, the reaction rate, the selectivity and the operating life of the catalyst and is therefore advantageously removed from the reaction product only in the work-up of this, e.g. by distillation.

After the reaction product mixture has advantageously been depressurized, the excess aminating agent and the hydrogen are removed therefrom and the amination products obtained (ethylamines) are purified by distillation or rectification, liquid extraction or crystallization. The excess aminating agent and the hydrogen are advantageously recirculated to the reaction zone. The same applies to any incompletely reacted alcohol.

The amines prepared using the process of the invention are suitable, inter alia, as intermediates in the preparation of fuel additives (U.S. Pat. No. 3,275,554; DE-A-21 25 039 and DE-A-36 11 230), surfactants, drugs and crop protection agents, hardeners for epoxy resins, catalysts for polyurethanes, intermediates for preparing quaternary ammonium compounds, plasticizers, corrosion inhibitors, synthetic resins, ion exchangers, textile assistants, dyes, vulcanization accelerators and/or emulsifiers.

All ppm figures in this document are by weight.

### EXAMPLES

### Example 1

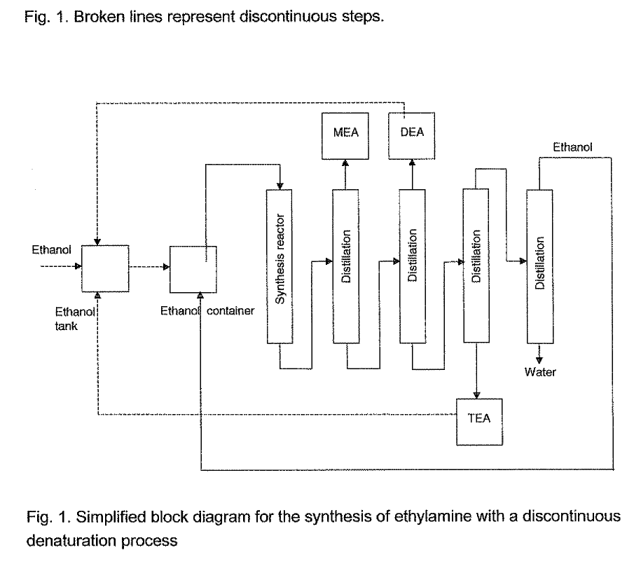

This example relates to the block diagram of FIG. 1 (appendix). The denaturation of the ethanol is carried out in a step which precedes the continuous ethylamine synthesis. 980 kg of bioethanol are introduced into an ethanol stock vessel (ethanol tank). 20 kg of DEA (diethylamine) are then metered in as denaturant from the DEA container so that the DEA concentration in the ethanol in the ethanol tank is 2.0% by weight. The EtOH/DEA mixture (denatured ethanol) is then pumped into the ethanol container for subsequent use for reaction with ammonia.

### Example 2

This example likewise relates to the block diagram of FIG. 1 (appendix). The denaturation of the ethanol is carried out in a step which precedes the continuous ethylamine synthesis. 980 kg of bioethanol are introduced into an ethanol stock vessel (ethanol tank). 20 kg of TEA (triethylamine) are then metered in as denaturant from the TEA container so that the TEA concentration in the ethanol in the ethanol tank is 2.0% by weight. The EtOH/TEA mixture (denatured ethanol) is then pumped into the ethanol container for subsequent use for reaction with ammonia.

1-19. (canceled)

20. A process comprising:
(a) providing an ethanol reactant and ammonia; and
(b) reacting the ethanol reactant with the ammonia in the presence of hydrogen and a heterogeneous hydrogenation/dehydrogenation catalyst to form a product comprising one or more ethylamines selected from the group consisting of monoethylamines, diethylamines, triethylamines and mixtures thereof;
wherein the ethanol reactant comprises ethanol denatured by the addition of a denaturant comprising an ethylamine selected from the group consisting of diethylamine, triethylamine and mixtures thereof.

21. The process according to claim 20, wherein the ethanol reactant comprises a biochemically or biologically prepared ethanol denatured by the addition of a denaturant comprising an ethylamine selected from the group consisting of diethylamine, triethylamine and mixtures thereof.

22. The process according to claim 20, wherein the ethanol reactant comprises an ethanol prepared by fermentation and denatured by the addition of a denaturant comprising an ethylamine selected from the group consisting of diethylamine, triethylamine and mixtures thereof.

23. The process according to claim 20, wherein the ethanol reactant comprises a synthetic ethanol denatured by the addition of a denaturant comprising an ethylamine selected from the group consisting of diethylamine, triethylamine and mixtures thereof.

24. The process according to claim 20, wherein the denaturant is added to the ethanol reactant in an amount of 0.01 to 50% by weight.

25. The process according to claim 20, wherein the denaturant is added to the ethanol reactant in an amount of 0.1 to 20% by weight.

26. The process according to claim 20, wherein the process is carried out continuously.

27. The process according to claim 20, further comprising subjecting the product to distillation to form a diethylamine product.

28. The process according to claim 27, wherein the denaturant comprises at least a portion of the diethylamine product.

29. The process according to claim 20, wherein the heterogeneous hydrogenation/dehydrogenation catalyst comprises a metal selected from the group consisting of Cu, Co, Ni, and mixtures thereof.

30. The process according to claim 20, wherein the heterogeneous hydrogenation/dehydrogenation catalyst comprises a support material selected from the group consisting of zirconium dioxide (ZrO₂), aluminum oxide (Al₂O₃), and mixtures thereof.

31. The process according to claim 20, wherein the heterogeneous hydrogenation/dehydrogenation catalysts prior to activation with hydrogen, comprises 20 to 85% by weight of Al₂O₃, 1 to 10% by weight of CuO, 5 to 20% by weight of CoO and 5 to 20% by weight of NiO.

32. The process according to claim 20, wherein the reaction is carried out at a temperature of 80 to 300° C.

33. The process according to claim 20, wherein the reaction is carried out in the liquid phase at a pressure of 5 to 30 MPa.

34. The process according to claim 20, wherein the reaction is carried out in the gas phase at a pressure of 0.1 to 40 MPa.

35. A continuous process comprising:
(a) providing an ethanol reactant and ammonia; and
(b) reacting the ethanol reactant with the ammonia in the presence of hydrogen and a heterogeneous hydrogenation/dehydrogenation catalyst to form a product comprising one or more ethylamines selected from the group consisting of monoethylamines, diethylamines, triethylamines and mixtures thereof, wherein the reaction is carried out at a temperature of 80 to 300° C., and wherein the heterogeneous hydrogenation/dehydrogenation catalyst comprises (i) a metal selected from the group consisting of Cu, Co, Ni, and mixtures thereof, and (ii) a support material selected from the group consisting of zirconium dioxide (ZrO₂), aluminum oxide (Al₂O₃), and mixtures thereof;
wherein the ethanol reactant comprises ethanol denatured by the addition of a denaturant in an amount of 0.1 to 20% by weight, the denaturant comprising an ethylamine selected from the group consisting of diethylamine, triethylamine and mixtures thereof.

36. The process according to claim 35, further comprising subjecting the product to distillation to form a diethylamine product.

37. The process according to claim 36, wherein the denaturant comprises at least a portion of the diethylamine product.

38. A method of denaturing ethanol, the method comprising: providing ethanol; and adding to the ethanol a denaturant comprising an ethylamine selected from the group consisting of diethylamine, triethylamine, and mixtures thereof.

39. The method according to claim 38, wherein the denaturant consists of one or more ethylamines selected from the group consisting of diethylamine, triethylamine, and mixtures thereof.
